(19) United States
(12) Patent Application Publication
(10) Pub. No.: US 2024/0013852 A1
(43) Pub. Date: Jan. 11, 2024

(54) ENCODING AN ASSEMBLY OF THREE-DIMENSIONAL HIERARCHICALLY ORGANIZED NANOPARTICLE ARCHITECTURES THROUGH CHROMATIC BONDS

(71) Applicant: THE TRUSTEES OF COLUMBIA UNIVERSITY IN THE CITY OF NEW YORK, New York, NY (US)

(72) Inventors: Oleg GANG, Setauket, NY (US); Brian MINEVICH, New York, NY (US); Hamed EMAMY, New York, NY (US); Shuting XIANG, New York, NY (US); Jason S. KAHN, New York, NY (US); Aaron MICHELSON, New York, NY (US); Kim KISSLINGER, New York, NY (US); Sanat KUMAR, New York, NY (US)

(21) Appl. No.: 18/217,812

(22) Filed: Jul. 3, 2023

Related U.S. Application Data
(60) Provisional application No. 63/359,570, filed on Jul. 8, 2022.

(51) Int. Cl. G16B 15/10; G01N 23/201; B82Y 30/00

(52) U.S. Cl. CPC G16B 15/10; G01N 23/201; B82Y 30/00

(57) ABSTRACT

The disclosed matter provides systems and methods for encoding an assembly of three-dimensional (3D) hierarchically ordered nanoparticle architectures through chromatic bonds. Through identification of the repeating mesovoxels including chromatic bonds and voxels, the presented disclose matter can allow for encoding the 3D architectures by using symmetries of mesovoxel, enable a compression of the information amount required for encoding.

### CROSS-REFERENCE TO RELATED APPLICATION

This Non provisional application claims priority to U.S. Provisional Patent Application No. 63/359,570, which was filed on Jul. 8, 2022, the entire contents of which are incorporated by reference herein.

### STATEMENT REGARDING FEDERALLY SPONSORED RESEARCH

This invention was made with government support under Grant Nos. DE-SC0008772 and DE-SC0012704, awarded by the US Department of Energy (DOE). The government has certain rights in the invention.

### BACKGROUND

The disclosed subject matter relates to the design of three-dimensional (3D) architectures, and encoding an assembly of 3D hierarchically ordered nanoparticle architectures.

Nanoscale components can be assembled through bottom-up assembly approaches that use the principles of shapes, interactions, and entropic effects. However, achieving 3D architectures with a hierarchical organization of nanocomponents remains a challenge. Hierarchical structures can lead to the tailoring of mechanical and optical properties using relatively simple components, and are promising for addressing certain problems in energy materials, catalytic, and purification applications.

In several fields, e.g., biotechnology, the power of hierarchical organization has been demonstrated, for example, in connection with the colors of species, bone strength, and tissue mass transport, via DNA-based systems using Watson-Crick bonds programmed to have a specific encoding (e.g., color). An outstanding issue is how to self-assemble a designed, hierarchically organized 3D architecture that can integrate multiple nanocomponent types as prescribed.

Using blocks with directional and encoded bonds, a complex architecture can be prescribed through the specific coordination of multiple blocks, despite only a next-neighbor character of interaction of each bond. Encoded DNA blocks can be used to assemble quasi-crystals, and colored interactions can aid the design of complex finite size and periodic organizations.

There is a need for developing a reliable and effective technique for encoding the assembly of 3D hierarchically organized nanoparticle architectures.

### SUMMARY

The disclosed matter provides techniques for generating 3D nanoparticle architectures, including specific structures through repeating motifs integrating chromatic directional bonds.

One aspect of the subject matter provides systems for encoding an assembly of a three-dimensional (3D) hierarchically ordered nanoparticle architectures. An example system includes a non-transitory storage medium having instructions of an encoding program stored thereon, and one or more data processing apparatus configured to run the instructions of the encoding program to perform operations.

In certain embodiments, the system can identify a target structure for the nanoparticle architecture, determine chromatic bonds for each voxel, encode the target structure by coordinating chromatic bonds within sets of voxels to create at least a mesovoxel, and generate an assembly by using a symmetry of the mesovoxels.

In certain embodiments, the system further includes an additive experimental device. The processing apparatus can output a structural indicator to the experimental device for verifying the assembly of 3D nanoparticle architectures. The experimental device can include an apparatus of Small-Angle X-ray Scattering, with the structural indicator being structural factor S(q).

In certain embodiments, the assembly can include a lattice having a lattice topology and lattice parameters. The voxel can be organized in a grid. The symmetry can include a rotation symmetry and/or mirror symmetry. A mesovoxel can include determining number of voxels, internal colors, and external colors.

Another aspect of the disclosed subject matter provides methods for encoding an assembly of a three-dimensional (3D) hierarchically ordered nanoparticle architectures. In certain embodiments, an example method can include identifying a target structure for the nanoparticle architecture, determining chromatic bonds for each voxel, encoding the target structure by coordinating chromatic bonds within sets of voxels to create at least a mesovoxel, and generating the assembly by using a symmetry of the mesovoxels.

In certain embodiments, the chromatic bonds include the internal and external bonds of each voxel. The voxel can be determined by using addressable interactions, where each bond has a specific encoding associated with both type and energy of interaction, while interactions of different colors are non-interacting. The voxel can include a DNA frame, for coordinating the placement of voxels within the mesovoxel. The voxel can be configured as a DNA origami octahedron. The target structure can include 1D strings, a 2D plane, a Face-Perovskite lattice, and a cubic lattice. The symmetry can be implemented by adapting mesovoxels into multiple repeated units.

In certain embodiments, the method can further include implementing an experimental verification of nanoparticle architectures using x-ray scattering and electron microscopy.

In certain embodiments, the nanoparticles can include gold nanoparticles, silver nanoparticles, magnetic nanoparticles, or any combination thereof.

Another aspect of the disclosed subject matter provides methods for designing a three-dimensional (3D) lattice of nanoparticles with nanoscale and photonic length scales. In certain embodiments, an example method can include identifying photonic regimes including periodicity, spacing, and separation of nanoparticles in the 3D lattice, determining chromatic bonds for each voxel based on the photonic regimes, encoding the nanoparticle architectures by coordinating chromatic bonds within sets of voxels to create at least a mesovoxel, and assembling the 3D lattice with coupled nanoscale and photonic lengthscales by using a symmetry of the mesovoxels. In certain embodiments, the symmetry includes a mirror plane symmetry, where the mirror plane bisects the voxels at the midpoint between the two planes of nanoparticles-filled planes, enabling the use of two colors for coordinating voxels in the Z-direction. The mesovoxel can include 6 unique voxels, 1 internal colored bond, and 5 external-colored bonds.

Another aspect of the disclosed subject matter provides methods for designing 3D lattice of ordered spiral nanoparticle motifs. In certain embodiments, an example method includes identifying a screw axis symmetry along Z-direction, designing a mesovoxel including N₁ voxels displaying N₂ external colors, where N₁ and N₂ are positive integers. A N₃×N₃ voxel arrangement can be rotated in the XY plane around the screw-axis along the Z-direction to generate a N₃×N₃ spiral, where N₃ is a positive integer. Nanoparticles can be incorporated into the voxels in the XY plane with specific particle placement and internal mesovoxel coloring. A rotation can be executed through external mesovoxel binding with coloring of vertices in the Z-direction possessing N₄ colors with respective complements thereof on neighboring voxels, where N₄ is a positive integer, and the N₃×N₃ spiral can be translated in the XY plane using further external mesovoxel coloring to enable encoding a 3D lattice of ordered spiral nanoparticles motifs.

In certain embodiments, N₁<4, and N₂<12. The ordered 3D lattice can be arranged to generate plasmonic and light-emitting chiral clusters for light manipulation. The assembled 3D lattice is arranged to generate a crystal of periodic, spiraling assemblies with a screw axis along the Z-axis.

### BRIEF DESCRIPTION OF THE DRAWINGS

The patent or application file contains at least one drawing executed in color, Copies of this patent or patent application publication with color drawing(s) will be provided by the Office upon request and payment of the necessary fee.

Reference will now be made in detail to the various exemplary embodiments of the disclosed subject matter, which are illustrated in the accompanying drawings. The accompanying drawings, where like reference numerals refer to identical or functionally similar elements throughout the separate views, serve to further illustrate various embodiments and to explain various principles and advantages all in accordance with the disclosed subject matter.

FIGS. 1A-1B provide a protocol of computing system and implementing process for encoding 3D hierarchically order nanoparticle architectures of the disclosed subject matter. FIG. 1A provides an illustrative diagram of a system to design and encode an assembly of 3D nanoparticle architecture of the disclosed subjects. FIG. 1B provides an illustrative process of encoding an assembly of 3D nanoparticle architecture of the disclosed subject matter.

FIGS. 2A-2B provide an illustration for the framework of the inverse design and assembly of hierarchically ordered 3D nanostructure through voxels encoded with chromatic bonds of the disclosed subject matter. FIG. 2A a provides schematic illustration of structural nanocomponents, including a chromatic voxel set, three chromatic binding descriptors, mesovoxel, and desired lattice. FIG. 2B provides a diagram of lattice grid, SEM image, and mesovoxel structure in some embodiments of the disclosed subject matter.

FIGS. 3A-3E provide schematic illustration of 3D encoded assembly for one-dimensional and two-dimensional hierarchically ordered nanoparticle structure of the disclosed subject matter. FIG. 3A provides a 3D assembly of 1D particle strings in an embodiment. FIG. 3B provides an SAXS comparison result of experimental structure factors S(q), shown in black, with S(q) shown in red, including associated locations of Bragg peaks and inset of the diffraction pattern in the embodiment of 1D particle strings. FIG. 3C provides a 3D assembly of 2D particle planes in another embodiment of the disclosed subject matter. FIG. 3D provides an SAXS comparison result of experimental S(q) shown in black, with S(q) for the model shown in red, including associated locations of Bragg peaks. FIG. 3E provides FIB section TEM imaging illustration of the select 2D plane assembly cross-sections.

FIGS. 4A-4C provide a schematic illustration of 3D encoded assembly for a nanoscale face-perovskite type lattice in another embodiment of the disclosed subject matter. FIG. 3A provides an internally origami color set, based on particle type, are mapped into a perovskite unit cell of 27 origami using a mesovoxel of 6 origami. FIG. 4B provides an SAXS comparison result of experimental structure factors, S(q), shown in black plots and modelled S(q) shown in red with associated Bragg peak locations and inset diffraction patterns, for the three ‘types’ of particles added individually, including (i) SC (conner), (ii) TEM image of SC, (iii) BCT, (iv) SC (center), where combinations of two particle types yield either (iv) FCC or (v) BCC, and addition of all three types yield a (vii) face-perovskite, and (viii) Perovskite FIB/TEM image. FIG. 4C provides a schematic illustration of modelled crystal growth of a DNA scaffold demonstrates lattice crystallization using (i) the hierarchical perovskite mesovoxel and (ii) no lattice formation with a fully-prescribed, 27-voxel cell, with plots in respective plots in (iii) and (iv) showing maximum crystallite size and number of crystal clusters as a function of time, in the embodiment of the disclosed subject matter.

FIGS. 5A-5E provide a schematic illustration of encoded assembly of 3D lattice with nanoscale (in-plane) and photonic (inter-plane) spacing regimes in another embodiment of the disclosed subject matter. FIG. 5A provides a schematic illustration of desired spaced-plane substructure and chromatic bonds layout. FIG. 5B provides a SAXS comparison result for synthesized lattice (black, experimental) versus modelled structure factor, S(q), shown in red with associated indexing of Bragg peaks and inset diffraction, FIG. 5C provides a SEM image of silicated lattice conveys large scale order, as well as maintenance of the simple cubic voxel organization chromatically scaffolding the tetragonal particle organization. FIG. 5D provides a TEM image of the same silicated lattice crystal sectioned by FIB viewing the (100) crystal face. FIG. 5E provides a diagram of Energy dispersive X-ray spectroscopy (EDS).

FIGS. 6A-6C provide a schematic illustration of encoding an assembly of a 3D lattice from spiraling nanoparticle motifs in another embodiment of the disclosed matter. FIG. 6A provides a schematic illustration of desired spiral NP motif and chromatic bonds layout. FIG. 6B provides an SAXS comparison result of experimental structure factor, S(q), black line, and a model S(q), red line with associated Bragg peak locations, and inset is a measured SAXS pattern. FIG. 6C provides an illustration of 3D lattice with arrangement of spiral NP motifs, as visualized from different crystal faces, with associated Miller indices referenced and angle of view schematically demonstrated, and particle unit cell corresponds to the space group in another embodiment of the disclosed subject matter.

It is to be understood that both the foregoing general description and the following detailed description are exemplary and are intended to provide further explanations of the disclosed subject matter.

### DETAILED DESCRIPTION

The disclosed subject matter provides a molecular array of contorted macromolecular ladders, preparation for same, and use in an electrode and lithium-ion battery. This molecular array can deliver a highly efficient performance when utilized in lithium-ion battery applications.

For clarity, but not by way of limitation, the detailed description of the disclosed subject matter is divided into the following subsections:

### I. Definition

### II. Experimental and Design Frame

### III. Examples

### I. Definition

As used herein, the term “design” refers to research and development of materials or structures thereof, e.g., via computer simulations, which can be implemented by several types of software on a computer. Design can include an investigation and predictions on behaviors of materials, especially nanoscale materials, where such behaviors include, but not limited to, composition, structure, and/or properties of materials.

As used herein, the term “encoding”, also known as DNA programming, refers to the process of using DNA molecules as a way to store and process information. This process involves using the several nucleotide bases in DNA (e.g., adenine, guanine, cytosine, and thymine) to represent bits of digital information, such as 0s and 1s. In DNA encoding, sequences of nucleotides can be used to represent specific pieces of information. In the field of nanomaterial engineering, hierarchical assembly can be encoded through nanoscale building blocks integrating DNA-related programmable bonds.

The term “voxel in lattice” refers to a three-dimensional unit cell or building block of a crystal lattice. In crystallography, a crystal lattice is a regular, repeating pattern of atoms, ions, or molecules that extends in three dimensions. The voxel is the smallest unit of a crystal lattice that can be repeated to generate the full structure. Each voxel in a crystal lattice contains one or more atoms, ions, or molecules, which are arranged in a pattern determined by the crystal's symmetry and structure. The shape and size of the voxel can vary depending on the crystal structure and the specific type of atom or molecule it contains.

As used herein, the term “chromatic voxel” refers to a small three-dimensional volume element that is associated with a specific color or chromatic property. Generally, chromatic voxels can be used to represent colors or textures of objects in a 3D scene. Each chromatic voxel can be assigned a specific RGB (e.g., red, green, blue) color value, or other color model, to represent the color or texture of a specific portion of the object. In other fields such as biotechnology, chromatic voxels can be used to represent different molecular properties or characteristics of biological systems, such as protein structures or cellular processes. By assigning different chromatic values to different molecular properties, it enables a visual display for the structure and behavior of complex biological systems in three dimensions.

As used herein, the term “mesovoxels” or “mesoscale motifs” refer to repeating patterns of structures or functional units that occur on an intermediate length scale between the nanoscale and the macroscale. These motifs can be observed in a wide range of natural and synthetic materials, including biological systems, polymers, and inorganic materials. In the context of materials science, mesoscale motifs are important because they can be used as building blocks for the design and assembly of more complex materials and structures. Examples of mesoscale motifs include helices, spirals, stripes, voxels, and block copolymers. These motifs can be used to create a wide range of materials with specific properties, e.g., three-dimensional hierarchically ordered nanoparticle architectures. In a design of the mesovoxel, several typical features, e.g., the numbers and types of voxels, the internally coloring, and externally coloring, are determined.

As used herein, the terms “chromatic bond”, “chromatic binding”, “programmable bond” or “programmable binding” refer to a type of DNA-encoded (“chromatic”) directional bonds used in the assembly of nanoparticles into hierarchically ordered 3D architectures. These bonds are formed by functionalizing nanoparticles with complementary DNA strands that can hybridize and form stable duplexes, allowing the nanoparticles to be linked together in a specific orientation. The term “chromatic” refers to the ability to use different colors of DNA strands to encode information about the orientation and directionality of the bonds between the nanoparticles. By designing the sequences of the DNA strands and the arrangement of the nanoparticles, it can foster control of directionality of the bonds and assemble the nanoparticles into complex 3D structures with specific properties and functions. The use of programmable bonds can have several advantages, e.g., being reversible and precisely programmed, allowing for precise control over the structure and properties of the assembled materials. Additionally, the use of programmable bonds can allow for the assembly of diverse types of nanoparticles, including with different sizes, shapes, and surface chemistries.

As used herein, the term “face-perovskite lattice” refers to a type of crystal structure that is composed of repeating units of face-centered cubic (FCC) and perovskite structures. The perovskite structure is a common crystal structure that is characterized by a cubic unit cell with a single cation in the center and an octahedral arrangement of anions surrounding it. The face-centered cubic structure is another common crystal structure that is characterized by a cubic unit cell with atoms located at each of the eight corners and the center of each face. In the face-perovskite lattice, the perovskite and FCC structures are interleaved in a pattern, resulting in a crystal structure with a variety of interesting properties. This lattice can be of interest in materials science because it exhibits a range of electronic, optical, and mechanical properties, including high-temperature superconductivity, high hardness, and high thermal conductivity.

As used herein, the term “Watson-Crick bond” refers to a type of chemical bond formed between two complementary nitrogenous bases in DNA molecules, namely adenine (A) and thymine (T), as well as between guanine (G) and cytosine (C). These base pairs are held together by hydrogen bonds, with A forming two hydrogen bonds with T, and G forming three hydrogen bonds with C.

As used herein, the term “Kern-Frenkel potential” refers to a theoretical model used to describe the interactions between particles, such as atoms or molecules. It is a type of intermolecular potential that can account for both the repulsive and attractive forces between particles. The Kern-Frenkel potential can be generally given by the equation or its derivative: V(r)=ε exp(−ar)−γexp(−βr), where r is the distance between particles, ε is the energy scale of the interaction, α and β are constants that determine the range of the repulsive and attractive forces, respectively, and γ is a constant that determines the strength of the attractive force. [

As used herein, the term “Monte Carlo simulation” refers to a computational method that uses statistical sampling techniques to model complex systems and processes. It is used in scientific research, engineering, and other fields to estimate the behavior of a system or process under different conditions. In Monte Carlo simulation, a large number of random samples or simulations can be generated to estimate the behavior of a system or process. The results can then be analyzed statistically to determine the probability distributions of the outcomes.

As used herein, the term “SAXS” refers to Small-Angle X-ray Scattering (SAXS), a technique used to study the structure of materials at the nanoscale. It involves shining a beam of X-rays at a sample and measuring the scattering pattern that results. This scattering pattern can provide information on the size, shape, and arrangement of the nanoscale structures within the sample. Relatively, the term “S(q)” refers to the scattering intensity as a function of the scattering vector q in SAXS. The scattering vector S(q) is related to the angle of scattering and the wavelength of the X-rays used. S(q) can provide information on the distribution of scattering particles within the sample. The model or modelled S(q) refers to calculation of model form factor for assembly including origami via theoretical computation.

As used herein, the term “FIB” stands for Focused Ion Beam (FIB), a technique used to selectively remove material from a sample at the nanoscale. It involves directing a beam of ions at a specific location on the sample surface, which can be used to create patterns or remove material for further analysis. FIB is a tool for TEM sample preparation that allows for the fabrication of electron-transparent foils from any region of interest (i.e., site-specific) and in virtually any material.

As used herein, the term “correct partner” refers to a lattice point or atom that interacts with another lattice point or atom in a way that is consistent with the desired crystal structure. In a crystal lattice, atoms are arranged in a regular and repeating pattern, with each atom interacting with its neighboring atoms in a specific way. For example, in a simple cubic lattice, each atom has six neighbors arranged in a cube-like structure. In a face-centered cubic (FCC) lattice, each atom has 12 neighbors arranged in a more complex pattern.

As used herein, the term “entropic penalty” refers to the energetic cost associated with reducing the entropy (or disorder) of a system. In other words, when a system transitions from a state of high entropy to a state of low entropy, energy is required to overcome the entropic penalty. The entropic penalty can be a fundamental concept in many areas of science and engineering, including chemistry, physics, and materials science. It arises in many contexts, such as in the crystallization of a liquid into a solid, the folding of proteins, and the ordering of magnetic domains in a material.

### II. Materials and Methods

Design, Synthesis, and Purification of Example DNA Origami.

The octahedron DNA origami frame was designed using the DNA origami design software caDNAano (http://cadnano.org/). The edges of the octahedron frame were designed to be six-helix bundles (6HB) with edge lengths of 84 base pairs. Each of the 6HBs have one sequence extending from each of the ends. This resulted in four ssDNA (single-stranded DNA) sequences extending away from each of the six vertices of the octahedron. For the capture and positioning of nanoscale components in the interior space of the origami, eight ssDNA sequences from four of the 6HBs were modified to have overhangs that extend toward the interior void of the origami.

DNA origami frames were folded by mixing 40 nM M13mp18 scaffold purchased from Bayou Biolabs, LLC with a 5:1 staple to scaffold ratio. The staple sequences were purchased from Integrated DNA Technologies (IDT). The buffer of this solution was mixed to contain 1 mM EDTA, 40 mM Tris at a pH of approximately 8.0, and 12.5 mM magnesium chloride. This mixture was heated to 90° C. and slowly cooled to 20° C. over a period of 20 hours to allow for the correct folding of the scaffold sequence into the target 3D DNA origami frame. This protocol can be adapted accordingly.

The folded DNA origami was washed several times to remove the excess staples used during the folding process. The origami samples were washed five times through a Millipore Sigma Amicon Ultra-0.5 mL centrifugal filters in an Eppendorf 5424R at a speed of 2.2 k rpm for minutes using a buffer consisting of (1×) TAE and 12.5 mM Mg²⁺.

Design of Example External and Internal DNA binding sequences.

The generation of a library of binding colors can create a non-interacting ssDNA set with similar melting temperatures and minimal formation of secondary structures. Design for external voxel binding sequences, which overhang from the octahedra vertices and include sequences in V^{m}b and V^{e}c, was undertaken according to the following:
1.) Each binding color sequence has 12 available bases, with the complement of that color possessing 8 bases. Thus, each binding interaction is hybridized through 8 bases. This design was selected to allow for modifications of binding strength, though in the disclosed subject matter, it used 8 bases for the complement.
2.) Approximately 50% G/C content in each strand.
3.) No greater than two consecutive G or C bases.
4.) In order to restrict base commonality and potential off-target binding, no more than 3 consecutive base matches allowed between different color strands, and no more than 4 total base matches in the 8 base binding regions.

Internal voxel binding sequences, described as Vⁱa, were designed as a separate set of 15 base binding sequences for the both the internal sequence color and its complement linked to the nanoparticle. G/C content was designed to be about a third of the binding bases, with no more than 2 consecutive G or C, and no significant hybridization within this set or the external voxel binding library.

Preparation of Example DNA functionalized Gold Nanoparticles (AuNPs).

Thiolated oligonucleotides, also purchased from IDT, were reduced by TCEP (tris[2-carboxyethyl] phosphine) at a 100× molar excess. Gold nanoparticles with a diameter of 10 nm, purchased from Ted Pella Inc., were mixed with the thiolated oligonucleotides at a ratio of 1:300. The mixture was buffered to be a 10 mM phosphate buffer (pH=7) after a 1.5-hour incubation at room temperature. After another 1.5-hour incubation, the sodium chloride concentration was slowly brought up to 300 mM over 250 minutes and then aged at room temperature overnight or for at least 12 hours. The functionalized gold nanoparticles were washed with distilled water, to remove the excess thiolated sequences and salt, five times in an Eppendorf 5424R at a speed of 15 k rpm. Dynamic Light Scattering (DLS) is used to track the change in the diameter of the nanoparticles with the addition of the DNA sequences to the surface.

Superlattice sample preparation.

An equimolar mixture of all of the origami was mixed in solution. Gold nanoparticles were then added to this mixture at a (2×) molar excess of the number of binding sites.

The mixture was heated to 50° C. and slowly cooled to room temperature over the course of five days at a rate of −0.2° C./hr. This protocol can be adapted accordingly.

Pre-loading of Origami with DNA-functionalized 10 nm AuNP's.

To provide for a greater percentage of nanoparticles loaded into the superlattices with several specific mesovoxel design, e.g., for spiral motifs, the origami designed to capture the particle was first annealed with (4×) excess of DNA-functionalized gold nanoparticles. These pre-loaded origamis where then mixed with the other DNA origami as a part of the respective designs and then annealed according to the protocol described in the section of “Superlattice sample preparation”. The pre-loading annealing protocol was the following: hold at 50° C. for 5 minutes, then ramp of −0.2° C./hr until 40° C., then hold at 40° C. for 5 minutes, then ramp of −0.6° C./hr until and hold at 25° C. for two minutes.

As demonstrated, the average filling of the octahedron DNA origami with 10 nm DNA-functionalized AuNP's was 74.1%.

Silication of DNA-NP Superlattices.

DNA origami superlattices were coated with robust silica through a sol-gel procedure for subsequent visualization in SEM/TEM. For conversion to inorganic silica, superlattices were centrifuged and supernatant replaced with (1×) Tris Acetate (TA) buffer pH 8.0, with 10 mM Mg Acetate (MgA).

The sol gel solution was prepared with 0.2-0.5% 3-Aminopropyl-triethoxysilane (APTES) and 0.7-2% solution of Tetraethoxysilane (TEOS), in (1×) TA with 10 mM MgA. The APTES was added first followed immediately by TEOS, which was then mixed vigorously at 500-800 rpm in a vortexer for 25-30 minutes. The solution was filtered with a Millex-GV PVDF 0.22 um um filters. 15 ul of filtered solution was mixed with 5 μl of superlattice and put back into a shaker at 700 rpm for 1-1.5 hours. The reaction was stopped by adding DI water and buffer exchanging the supernatant 2 times with DI water. The silica-superlattice conjugate was drop-cast to a silicon chip substrate for observation in SEM. Or onto a 300 mesh TEM grid for TEM.

Sample preparation and electron microscopy (TEM and SEM).

Samples were drop cast from solution to either a silicon wafer or 300 mesh TEM grids and dried at RT for observation in SEM and TEM. SEM was performed using the Hitachi 4800 SEM, typically operation conditions were 3KeV and 7-10 nA current. Transmission electron microscopy was performed using a Jeol 1400 operated at 120 KeV, with samples of the technique shown in perovskite imaging as embodied below.

Serial sectioning TEM/STEM/EDS.

BFTEM (bright field transmission electron microscopy)—the gray scale is roughly inversely proportional to the density of the sample material (vacuum is white and gold would be black).

HAADF STEM (high angle annular dark field scanning transmission electron microscopy)—the gray scale is proportional to atomic number (e.g., gold is white). All EDS acquisition took place in HAADF STEM mode.

TEM cross-sectional samples (TEM lamellae) were prepared using the in-situ lift-out method in the Helios 600 dualbeam FIB with final Ga+ (Gallium) milling at 5 keV to reduce beam damage.

Serial sectioning SEM and reconstruction/analysis of spiral lattice domains.

DNA-NP superlattices were drop cast onto a clean silicon wafer. Imaging and serial sectioning were performed using the FEI Helios Nanolab 660 SEM/FIB; Auto Slice and View G3 software suites was used to automate collection. The particle cluster was imaged with an Elstar in-lens BSE detector (TLD-BSE) at 5 KeV with a 10 μs dwell time and 100 pA current. A sacrificial layer of Pt was deposited on top of the sample to help avoid damaging the sample before slicing, after which a trench was milled surrounding the sample and the fiducial marker was placed with Pt for Auto slice and view functionality. The focused ion beam current was set to 0.24 nA at 30KeV, a 10 nm step size for layer removal was selected. A total of 162 slices were collected in the disclosed subject matter. Dragonfly ORS software was used to perform corrections and filter and view data.

Analysis of reconstructed 3D crystalline domains of the periodic spiral array used Dragonfly ORS software. The frames were aligned using cross correlation and particle positions were refined from the raw data through thresholding, followed by sobel filtering and open and close operations to minimize noise from pixels too small to be the nano-particles and as a separation operation to help clearly distinguish particle positions. A distance mapping filter was used to assist in equalizing contrast to aid visualization. Lastly, a subset of the data was boxed off and colorized red to visualize the lattice in the figures of the disclosed subject matter from different crystallographic orientations. In the microstructural image formation process, the nanoparticles are seen in the superlattice coated with Platinum for protection from the Gallium beam and the post processed image which highlights the nanoparticles of the superlattice.

Small-Angle X-ray Scattering (SAXS).

(a) Experimental Methods

The SAXS measurements were done at the Complex Materials Scattering (CMS) and Soft Matter Interfaces (SMI) beamlines at the National Synchrotron Light Source II (NSLS-II) at Brookhaven National Laboratory (BNL) in Upton, NY. The 2D scattering data was collected on area detectors located downstream of the samples' position.

TABLE 1
NSLS-II User End Station Experimental Setups

| Beamline | 11-BM CMS | 12-ID SMI |
|---|---|---|
| Photon Energy (keV) | 13.5 | 16.1 |
| Horizontal × Vertical Beam size | 200 × 200 | 20 × 200 |
| (μm × μm) | | |
| Approximate Flux (photons/sec) | 10 11 | 1012-1013 |
| Sample -to-Detector Distance (m) | 5.05 | 8.3 |
| Detector Manufacturer | Dectris | Dectris |
| Detector Model | Pilatus 1M | Pilatus 1M |
| Detector Pixel Size (μm × μm) | 172 × 172 | 172 × 172 |

The resultant area images of X ray scattering were integrated into a one-dimensional (1D) I(q) scattering curve as a function of the scattering vector q, where

$$q = 4 \frac{π}{λ} sin ( \frac{θ}{2} )$$

with λ and being the wavelength of the incident X-rays and the full scattering angle, respectively. The resultant 1D curves spanned roughly 0.04 nm⁻¹ to 1 nm⁻¹ with a resolution of 0.002 nm⁻¹. The structure factor S(q) was obtained by dividing I(q) by the corresponding particle form factor P(q).

In certain embodiments of the disclosed subject matter, modeling of the analysis can be performed using the ScatterSim software package, a python package that implements a scattering formalism for superlattices. This formalism can generically model the scattering from arbitrary anisotropic nano-objects within the unit cell of a regular superlattice. This library can be extended to perform more complex modeling, and code is available, e.g., for download on github (URL: https://github.com/CFN-softbio/scattersim).

There are only small deviations between the form factor calculations for the gold nanoparticle and the AuNP-Origami conjugate due to the large difference in scattering length densities (SLD) between gold and dsDNA.

The resultant scattering intensity, I(q), of any object in solution in q-space is directly related to the Fourier transform of the spatial arrangement of the electrons.

Computational Methods.

Methods

A hard octahedron (side-length=√2) is utilized to represent the octahedron. Similar to the experiments, there are bonds on each vertex of the octahedron. For simplicity of the model, it can use a single bond on each vertex and adjust the bond energy to account for 4 DNA bonds in the experimental system. These bonds are positioned at (±1.0, ±1.0, ±1.0) relative to the center of the octahedron. To mimic the DNA bonds between origami, the Kern-Frenkel potential can be applied as follow:

$$U = {U}_{r} ( r ) {U}_{ω} ( θ , {n}_{i} , {n}_{j} ) \qquad ( 1 )$$

$$Where , {U}_{r} ( ripjq ) = \{ \begin{matrix}- ε , if {r}_{ipjq \leq ϕ} \\ 0 , otherwise\end{matrix} \qquad ( 2 )$$

$${U}_{ω} ( θ , {n}_{i} , {n}_{j} ) = \{ \begin{matrix}d {δ}_{cpcq ,} if {n}_{ip} \cdot {r}_{ipjq} \geq cos ( {θ}_{0} ) and - {n}_{ip} \cdot {r}_{ipjq} > cos ( {θ}_{0} ) \\ 0 , otherwise\end{matrix} ω \qquad ( 3 )$$

where Uᵣ(ripjq) is a unit vector in the direction that connects the p^{th} linker (vertex) of the i^{th} octahedron to the q^{th} linker of the j^{th} octahedron, d is the bond-length, δ_{cpcq} is Kronecker delta, nᵢₚ is the direction that connects the center of the i^{th} octahedron that the p^{th} vertex of the same octahedron. The δ_{cpcq} (cp and cq are the colors of the p^{th} and q^{th} vertex respectively) term ensures that each color on cube i can potentially be bonded with just the corresponding color on the octahedron j and θ₀=0.2. The variable ∈ values are used and the growth of the lattice is investigated.

To have more information about the whole system, including the particles belong to smaller clusters, the disclosed subject matter can implements modelling of the normalized enthalpy of the system as a function of the simulation time. For a complex system the enthalpy increases, but the total energy of the system does not saturate to a final value during the simulations. However, for a simple system, the energy increases and saturates at around H=0.8. These results suggest that the growth rate of the simple system is significantly faster than the more complex system, and adds additional evidence that simpler design can lead to larger structures and faster growth rate.

Computing System and Process.

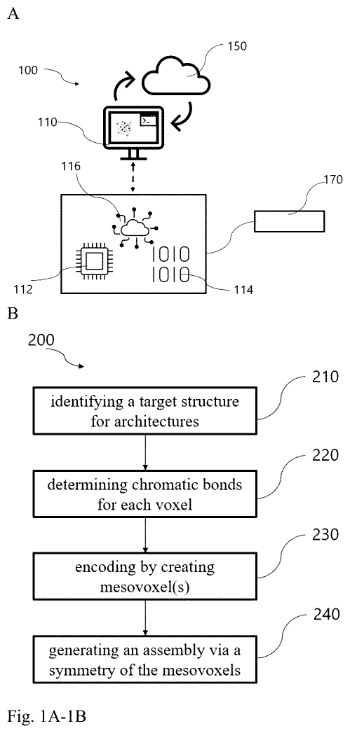

The protocol of computing system and process of the disclosed subject matter is shown in FIGS. 1A-1B.

FIG. 1A shows an example of a system 100 usable to design and manufacture lattice structures. A computer 110 includes at least a processor 112 and a memory 114, and the computer 110 can be connected to a network 150, which can b e a private network, a public network, a virtual private network, etc. The processor 112 can be one or more hardware processors, which each can include multiple processor cores. The memory 114 can include both volatile and non-volatile memory, such as Random Access Memory (RAM) and Flash RAM. The computer 110 can include various types of computer storage media and devices, which can include the memory 114, to store instructions of programs that run on the processor 112. Such programs can be implemented to perform operations to generate the assembly for 3D hierarchically ordered nanoparticle architectures via encoding mesovoxels.

Such programs can include an encoding program 116 for designing an assembly for 3D hierarchical order nanoparticle architecture, where the encoding program 116 can run locally on computer 110, remotely on a computer of one or more remote computer systems thereof (e.g., one or more third party providers' one or more server systems accessible by the computer 110 via the network 150), or both. The encoding program 116 can present a user interface (UI) on a display device of the computer 110, which can be operated using one or more input devices of the computer 110 (e.g., keyboard and mouse). In certain embodiments, the display device and/or input devices can also be integrated with each other and/or with the computer, such as in a tablet computer.

An operator can utilize the system 100 to create an encode an assembly of 3D hierarchical order architecture through performing certain operations on the computer 110 upon the implementation of the programs. The target architecture includes, but not limited to, strings, planes, cubes, or other structures. The operations on the computer 110 can include the modelling or computation of the target materials or structures.

In certain embodiments, multiple 3D nanoparticle architectures can be designed and assembled using a repeatable unit, mesovoxel. For example, with such repeatable unit in modeling, there is an opportunity to design and approach intrinsic symmetries of the mesovoxels of targeted 3D architectures, thereby reducing the amount of information required for encoding bonds.

The encoding program 116 can be programed to provide one or more user interface elements that enable the operator to customize mesovoxels for the 3D nanoparticle architectures. The mesovoxels customization via the encoding program 116 can be regulated using several types and numbers of voxels, chromatic binding/bonds. Additionally or selectively, the encoding program 116 can tailor multiple lattice topology types for lattice creations, including, but not limited to, BCC (body-centered cubic), FCC (face-centered cubic), BCT (Bod ofy-centered tetragonal), or any combination thereof. Each topology type can have a unique structural behavior, including unit size, lattice orientation, space distance, and angle in a space.

In certain embodiments, the system 100 further can include an experimental device 170, for an experimental verification. An exemplary device for verifying the target 3D hierarchical architectures encoded by the above programs can include a X ray scattering apparatus, e.g., an SAXS (small-angle X-ray scattering) apparatus. The target hierarchical architectures by encoding can be verified using a XAXS technique via the comparison of the structure factor, s(q).

FIG. 1B shows an exemplary process 200 of encoding the 3D hierarchically ordered 3D architectures of the disclosed matters. In certain embodiments, this process can be implemented upon the implementation of encoding program 116 by one or more computer system 100.

At 210, a target structure for the desired architectures is identified. Here, the lattice topologies, materials types, unit size can be identified for further computation and design. At 220, for each voxel, the pattern of chromatic bonds is determined as elements of a mesovoxel. For example, the chromatic binding types and colors are determined. At 230, a coordination of chromatic bonds within set of voxels is performed to create mesovoxel(s) for encoding. Specifically, certain featured behaviors of mesovoxels, e.g., the types and numbers of voxels, internal and external colors, can be established. At 240, an assembly for the target structure is generated through a symmetry of the mesovoxels. Notably, by operating the symmetry of mesovoxels, the required information for encoding the desired structure can be reduced significantly.

Design Framework.

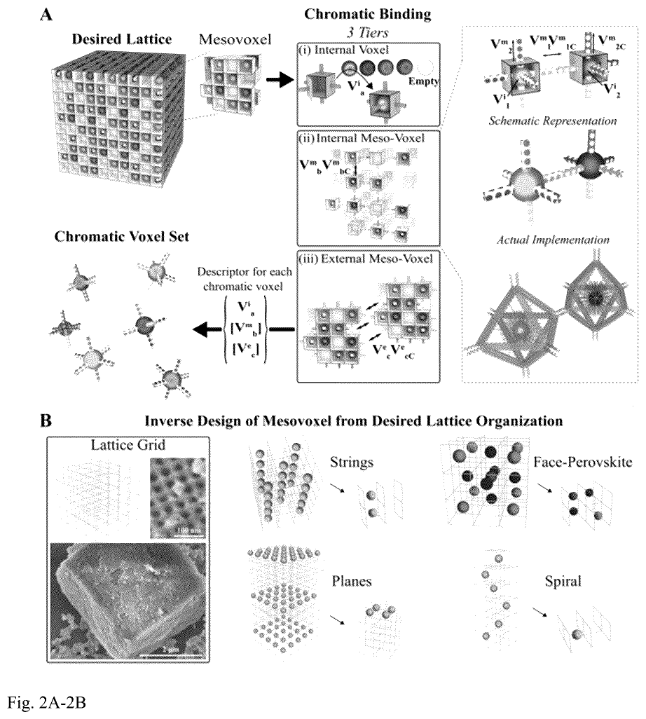

Herein, the disclosed subject matter provides a general inverse design framework for the assembly of targeted, ordered architectures (“blueprint”) through a set of components with specifically encoded interactions, whereby this set contains information about the equilibrium state of the blueprint. The approach can reduce the amount of information required for the encoding of the desired structure by accounting for intrinsic symmetric elements, thus, which is termed as an information-constrained assembly. On the other hand, an experimental demonstration has been implemented to confirm the application of the proposed strategy to design, encode, and assemble 3D hierarchically ordered organizations (FIG. 2A) and apply it to several exemplary cases (FIG. 2B) of nanoparticle lattices by creating: (i) low dimensional organizations with plasmonic and photonic scales in different crystal directions, (ii) complex yet designed face-perovskite lattice and (iii) hierarchically prescribed lattice assembled from spiral motifs.

In exemplary embodiments described here, the disclosed solutions have addressed a challenge of nanoscale manufacturing by providing a method for creating designed, three-dimensional (3D) nanomaterials on demand with specifically prescribed structure and composition using inverse design approaches. The disclosed subject matter utilizes of addressable interactions, where each bond has a specific encoding associated with both type and energy of interaction, while interactions of different colors are non-interacting. The disclosed subject matter uses DNA frames based on DNA origami constructs integrated with nano-components to form material voxels and lattices, allowing for the assembly of complex 3D architectures from nanoparticles with hierarchical organization. The advantageous result of the disclosed matter is demonstrated through several exemplary cases of nanoparticle lattices with different crystal directions, complex face-perovskite lattice, and hierarchically prescribed lattice assembled from spiral motifs.

FIGS. 2A-2B illustrates an exemplary framework of the inverse design and assembly of hierarchically ordered 3D nanostructures through voxels encoded with chromatic bonds. As illustrated in FIG. 2A, the elementary building block is a material voxel configured to be structured as an octahedron, that can either contain a nanocomponent or remain empty. Notably, the framework sets an octahedron frame as the voxel using vertex bonds, which results in a cubic grid. In certain embodiments, other voxel geometries, e.g., sphere, icosahedron, dodecahedron, tetrahedron, etc., can be utilized for creating structures within grids of different symmetries. As embodied herein, the DNA octahedron is formed from 12 edges, each consisting of 6 interlinked bundles of DNA, while intra-voxel binding of nanocomponents is achieved by 8 identical binding single stranded (ss) DNA “sticky end” strands within the same voxel. Herein, by using gold nanoparticles (AuNPs) grafted with complementary strands as an illustrative model. Inter-voxel chromatic bindings are determined by 4 ssDNA overhangs at the vertices, where a colored bond is determined by specific ssDNA and its complement on another frame, wherein the embodiment excludes the placement of a color bond and its complement on the same voxel. To simplify the nanostructural configuration, FIG. 2A presents the voxels and nanoparticles with a ball-and-stick schematic, where internal voxel binding colors are represented by particle color and external voxel binding color by sticks of a dotted color pattern, the complement of which is the inverse color pattern.

The repetitive motif of the target structure in the embodiment is presented as “mesovoxel”. To prescribe and encode a target structure, three tiers of color (‘chromatic’) binding descriptors are the following: Vⁱa defines each voxel's internal binding color for hosting a correspondingly encoded nanocomponent, and V^{m}b and V^{e}c define a voxel's external bonds. For each voxel type, V^{m}b defines a chromatic valence, then a set of V^{m}b interactions for all voxels forms a set which can encode local chromatic bindings of each voxel with 6 next-neighbor voxels. Through coordination of chromatic bonds V^{m}b within a set, a mesovoxel can be prescribed. A set of V^{e}c interactions defines how mesovoxels are bound to each other and the hierarchical order. The inverse design approach uses local symmetries present in the target architecture to minimize the number of unique voxels and chromatic bonds required to encode a mesovoxel, thus simplifying the matrix of voxel descriptors.

Notably, as shown in FIG. 2A, a desired 3D lattice of nanocomponents is constructed from material voxels, consisting of a DNA frame, that provides inter-voxel colored binding and valence-defined coordination, as well as encoded encapsulation of internally-bound components. Experimental implementation uses a DNA origami octahedron as the voxel, and gold nanoparticles (NP) functionalized with different ssDNA coronas as the internally-bound nano components.

FIG. 2B illustrates a schematic range of 3D NP lattices in an explosive view in certain embodiments, all synthesized by implementing chromatic binding in a simple cubic grid built from DNA octahedra, as shown in a SEM image. NP organizations are shown next to mesovoxel structures thereof. Exemplary embodiments for assembling strings, planes, face-perovskite, and spiral hierarchically ordered 3D nanostructures have been presented as below.

### III. Examples

### Example 1: Low-Dimensional Nanoparticle Organizations within a 3D Volume

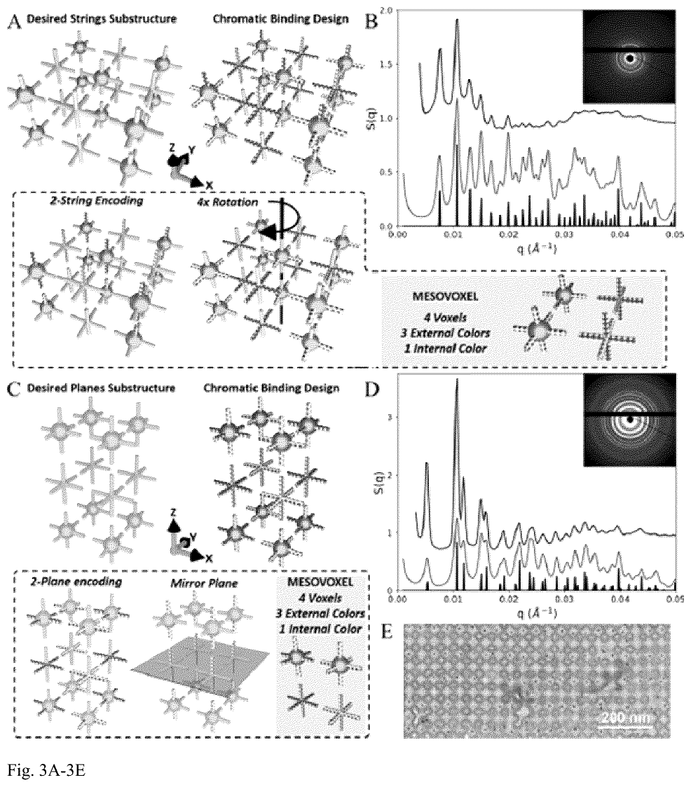

In the embodiment, it demonstrates 3D materials, characterized by different structures, with designed and formed organized low-dimensional nanoparticle (NP) arrangements, e.g., strings and planes, in 3D volume. This demonstration of hierarchical structuring targets breaks isotropic spacing in the grid through string and plane NP organizations. As illustrated in FIGS. 3A-3E, one-dimensional and two-dimensional hierarchical nanoparticle structuring are encoded and formulated utilizing different arrangements of the same color set.

As embodied herein, 3D arrays of ordered 1D nanoparticle “strings” and stacked 2D “planes”, as shown in FIG. 3A and FIG. 3C respectively, are designed within the simple cubic grid, as defined by the smallest center-to-center distance between voxels. In comparing the desired nanoparticle motifs to potential mesovoxel arrangements, the number of required voxels can be reduced by making use of motif symmetries advantageously. For example, the ‘string’ design is a 3D array of alternating NP-filled and empty 1D arrays. There is a single particle type in this lattice design which necessitates one internal binding color. This design, if defined with unique voxels, would require 54 unique external colors and 18 voxels. However, the use of symmetries can compress the amount of information required to prescribe the target structures. A single 1D array that repeats along the Z-axis can be made by one external binding color and its complement, and an additional string will require the use of another external binding color and complement. Connections between the alternating strings can be achieved through an additional chromatic bond coordinated using the four-fold symmetry of the remaining vertices in the XY-plane. Thus, the resultant meso-voxel is comprised of 4 voxels with 1 internal color and 3 external colors.

3D assembly of 1D particle string.

FIG. 3A illustrates a schematic diagram of a 3D assembly of 1D particle strings, where origami particles bind at a coordination of 180°. In the embodiment, nanoparticle strings are produced by the aforementioned 4 origami mesovoxel set, with two colors, e.g., green and red, placed along the Z-axis. Color 3, e.g., blue, and its complement are coordinated in the X-Y plane with a valence of 4, utilizing 4-fold rotational symmetry about each of the parallel voxel strings. The resulting architecture yields two representative particle spacings: a smaller NP spacing found within the strings and a larger NP spacing spanning strings. Structure factor, S(q), obtained from small angle x-ray scattering (SAXS pattern, as shown in FIG. 3B) confirms that the NPs are assembled as designed, and the SAXS signature of this design is a primitive tetragonal crystal lattice, where intra-string and inter-string spacings are 59.1 nm and 83.6 nm respectively. In addition, the model corresponds to a tetragonal primitive (tP) unit cell with lattice parameters a=b=83.59 nm, c=59.12 nm, α=13=γ=90°, upon measurement and calculation.

3D assembly of 2D plane.

Alternatively, in another embodiment, a different symmetry element is utilized to adjust alternating planes of NPs and empty voxels, organized in a 3D grid, as shown in FIG. 3C. FIG. 3C illustrates a schematic diagram of a 3D assembly of 2D particle planes, where particles bind at a coordination of 90° with a valence of 4, further separated along the Z-axis by an empty voxel layer. The 2D plane only requires 1 internal color because only a single nanocomponent is being used for this structure. The originally fully prescribed design would need 12 unique voxels with 36 external colors and 1 internal color. In contrast, as embodied herein, utilizing symmetries present in the target structure enables an effective reduction of the information required to prescribe this lattice. Notably, the two alternating planes will each use a separate color with 4-fold rotational symmetry to denote the separate planes, and the presence of a mirror plane symmetry in Z-direction allows the use of a third color to connect the planes. The mesovoxel in the embodiment consists of four unique voxels with 1 internal and 3 externally colored bonds. This nanostructure has a signature of a primitive tetragonal crystal phase confirmed by SAXS, as shown in FIG. 3D. Upon the measurement and calculation, the smallest NP spacings, 59.1 nm, are the same as those in the 1D assemblies, as determined from modelling of the SAXS, results yet are now present due to nearest neighbors of NPs in the XY plane. A larger NP spacing of 118.2 nm represents the distance between stacked 2D planes in the Z-direction. Using a lattice silication can mineralize the lattice, locking the DNA scaffold particles into place and allowing for FIB sectioning followed by TEM imaging of select assembly cross-sections. The modelled inter-plane particle spacings are confirmed by TEM real-space imaging shown in FIG. 3E.

### Example 2: Nanoscale Face-Perovskite Lattice

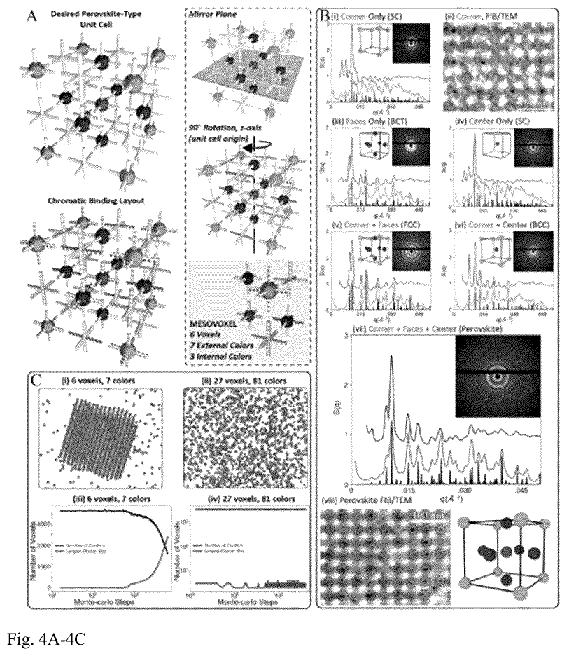

FIG. 4A-4C illustrate a design and assembly of a nanoscale face-perovskite type lattice. As embodied herein, A face-perovskite unit cell has three NP types—center (red), face (blue), and corner (green). Based on these particle types, the perovskite organization can be hierarchically arranged based on particle addition.

This embodiment has demonstrated that the mesovoxel methodology of the disclosed subject matter can also be applied to explicitly design for a desired crystallographic unit cell. As embodied herein, a target architecture, a nanoscale face-perovskite lattice, is designed, as shown in FIGS. 4A-4C. To define such a complex unit cell, a fully prescribed design would require the use of 27 distinct voxels and 81 external binding colors, but both values can be reduced through mesovoxel construction. The target unit cell possesses three unique particle locations and spacings between these particles: a body-centered particle (red), a face-centered particle (blue), and a corner particle (green), each requiring a separate internal color. To implement the assembly of Nanoscale face-perovskite lattice, the solution includes three internal colorings for each of these three particle types. By utilizing two symmetries in the mesovoxel approach, specifically a four-fold, Z-axis rotational symmetry about the central red particle, and mirror symmetry in the X-Y plane bisecting the unit cell, the nanoscale face-perovskite lattice has been designed with an effective decrease of voxels. This approach allows to achieve a mesovoxel design with 6 unique voxels and 7 external-colored bonds, in conjunction with the 3 internal colors.

In details, combinatorial filling of the colored lattice can be accomplished through addition of the three different particle types, each functionalized by a different ssDNA complementary to the set of 3 internal colors. It enables a production of crystalline subphases of the perovskite structure, which can be identified as FCC, BCC, BCT, and SC phases using SAXS probing of assemblies, as shown in FIG. 4B (i)-(vi). Assembly of a system with all three encoded particles realizes the designed face-perovskite ordering, as shown in FIG. 4B(vii). TEM imaging of the lattice containing only unit-cell corner (green) particles and the perovskite provide real-space confirmation of these particle organizations, with further images included in FIG. 4B(viii). SAXS confirms the successful assembly of all designed perovskite lattice and sub-phases. In this manner, a comparison of the perovskite, a lattice constructed by 6 different origami and 7 external voxel colors, to a simple-cubic particle lattice organized by two voxels and one external color, is made. There is a slight shift (<0.5 nm) in the lattice parameter, and a slight reduction in peak resolution in the high q-space region, though this difference is not significant when considering overall crystalline order, which confirms that crystal quality is not directly affected by the mesovoxel assembly strategy of the disclosed subject matter.

### Example 3: Cubic Particle Lattice

Notably, the number of unique voxels and colored bonds can significantly affect the assembly process due to the differences in energies of colored bonds and entropy factors associated with multiple types of voxels and multiple types of bonds in the systems. To further explore these effects of hierarchical assembly of target lattice compared to the fully prescribed counterpart, another embodiment performs molecular simulations on two designs of a simple cubic particle lattice—one using the chromatic assembly of the motif used in the perovskite lattice discussed above and the other using a fully-defined system with 27 distinct voxels and 81 external binding colors. All voxels were defined as possessing an internal NP, and an octahedron hard object was used as a proxy for the DNA frame, while bonds between vertices were modelled with the Kern-Frenkel potential. Using a Monte-Carlo simulation of hard polyhedral (Software, .HOOMD-blue package), the solution herein simulated the two associated mesovoxels (6 voxels per mesovoxel Vs. 3×3×3=27 voxels per mesovoxel). FIG. 4C shows that within the simulation time frame, the unit cell defined from 27 unique voxels remains a composed of individual voxels or small clusters, while designing mesovoxel system crystallizes into a large lattice. The behavior of crystallite size as a function of time implies slower crystal initiation versus crystal growth parameters for the mesovoxel system. While it can be slower, this process enables the growth of larger crystals with fewer defects. Furthermore, the total system energy for the fully-defined lattice is sustained at a significantly smaller value. Thus, larger numbers of unique voxels and bonds result in a decrease of the lattice quality and domain sizes, as both the likelihood of interaction with the correct partner decreases and the entropic penalty of crystallization increases.

### Example 4: Designing 3D Lattices with Coupled Nanoscale and Photonic Lengthscales

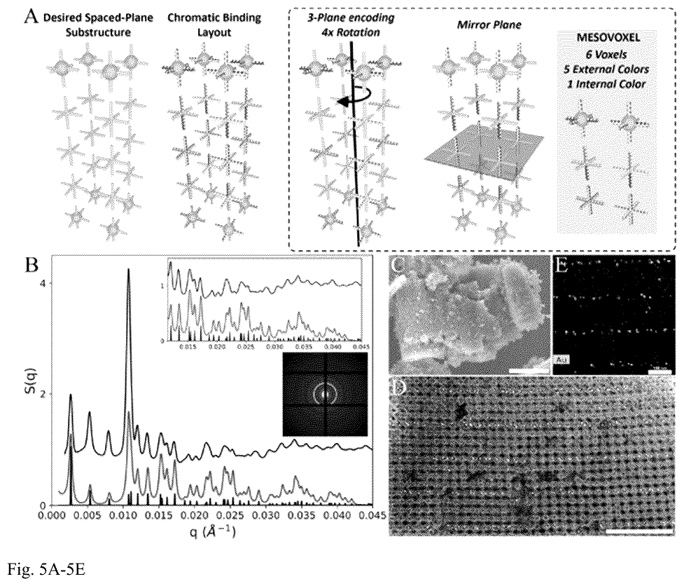

In this embodiment, an optical application regarding the methodology of designing 3D lattices of the disclosed subject matter is demonstrated, where the nanoscale optical effects with photonic regimes for applications in light emissions, information processing, and control of light have been demonstrated. In operation, it explores the use of the design strategy to realize 3D lattices with these two different lengthscale regimes arranged orthogonally. Photonic regimes require a periodicity of approximately half the wavelength, and thus a NP spacing of hundreds of nm is required, while nano optical effects need <tens of nm separation. The demonstration expands the design of 2D NP arrangements, ‘planes’ within 3D space, to interplanar spacing equivalent to 5 connected voxels, as shown in FIGS. 5A-5B. Such a target design would require 20 unique voxels and 60 externally colored bonds if fully defined previously. However, using the symmetry of the target lattice described above, the number of distinct voxels and bonds can be substantially minimized: a mirror plane symmetry that bisects the voxels at the midpoint between the two planes of NP-filled planes allows for the use of two colors for coordinating voxels in the Z-direction, which yields only three distinct layers in the design, with each needing a separate color to organize the voxels in the XY-plane, as shown in FIG. 5A. Upon the mechanism, the mesovoxel for this architecture consists of 6 unique voxels, 1 internal colored bond, and 5 external colors.

The expected large photonic spacing between NP planes yields a characteristic S(q)

peak in very low q-space, as shown in FIG. 5B, corresponding to a photonic distance of 233.4 nm, while intra-planar NP spacing is 58.3 nm, as revealed by SAXS analysis of the assembled lattice. This result is further confirmed by SEM imaging of the silicate 3D lattice. SEM image, as shown in FIG. 5C (Scale bar: 100 nm) demonstrating a 3D simple cubic grid of voxels, while TEM, illustrated FIG. 5D (Scale bar: 200 nm), confirms a SAXS conclusion about NP arrangements by showing (100) crystal plane with NPs placed within planes separated by three empty voxels. This structure corresponds to a tetragonal lattice of NPs with a high aspect ratio (˜4). The planes of gold NPs are also revealed by energy dispersive X-ray spectroscopy (EDS) TEM imaging, as shown in FIG. 5E.

### Example 5: Designed Organization of Ordered Spiral Motifs

In this embodiment, it designs and assembles complex hierarchical and ordered 3D organization. To illustrate, a lattice is designed, as to where the hierarchical motif is a spiral arrangement of NPs, to develop plasmonic and light emitting chiral clusters for light manipulation. This embodiment has achieved a rationally assemble of a 3D lattice form the spiral NP motif.

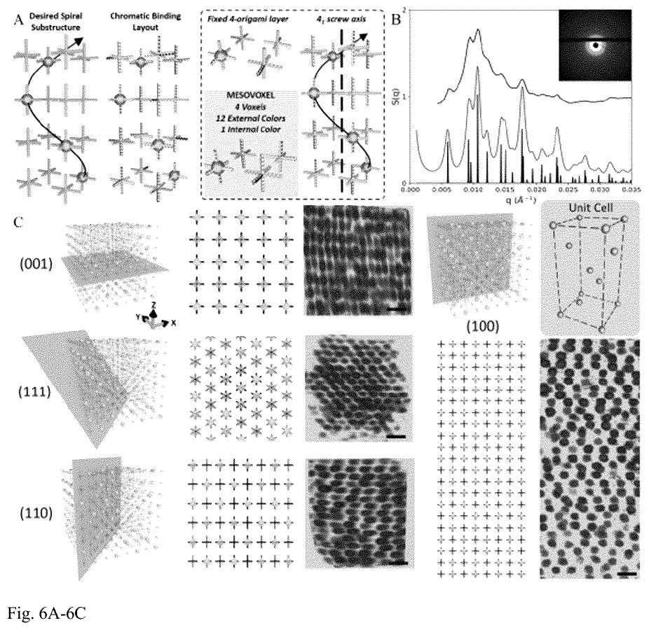

Notably, it demonstrates a crystal of periodic, spiraling assemblies by considering that the screw axis symmetry is inherent to this target organization, as shown in FIG. 6A. If fully-prescribed previously, this would require 16 unique voxels and 48 external colors. In this embodiment, information compression for this design has been achieved by identifying a 41 screw axis symmetry along the Z-direction. The screw axis symmetry, and the choice of one particle type, informs the mesovoxel design of this embodiment.

First, a 2×2 voxel arrangement in the XY plane can be rotated around the screw-axis; the need for this 4-voxel layer to retain its specific particle placement means that each neighbor binding can be specifically defined with internal mesovoxel coloring. In order to induce this rotation through external mesovoxel binding, the coloring of vertices in the Z-direction possesses 4 colors that with their respective complements on neighboring voxels induce a 90° rotation upon mesovoxel stacking along the Z-axis. Further external mesovoxel coloring enables the translation of the 2×2 spiral in the XY plane. This coloring can conceptually be achieved with a lower number of voxels and colors, but with greater tradeoffs in binding specificity that can negatively affect a crystal growth due to disordered presentation of binding colors along the screw axis. Herein, the 4 additional colors retain greater orientational specificity during voxel incorporation into the growing crystal.

The above presented strategy in the embodiment for defining the mesovoxel reduces the design to 4 distinct voxels with 12 colored bonds. The SAXS obtained S(q) for the assembled lattices (black line) based on this design match well the modeled 3D superlattice (red line) with a periodic organization of spiraling NPs motifs, as shown in FIG. 6B. This example identifies the formed structure as a unit cell with space group I4₁22. Notably, while the assembly motif is spiral, the formed lattice is not chiral since a close XY placement of the motifs in the lattice cancels the chirality in the overall structure. Compared to simpler designs, the complicated lattice, formed through the placements of spiral NP motifs, exhibits a broadening of diffraction peaks, and the number of higher-order peaks is decreased. In addition, SAXS results from an empty lattice show a similar behavior, which indicates that external voxel bonds play roles in this decreased degree of order. This effect is related to the larger number of externally colored bonds required for this design, and this observation is supported by modeling conclusions, as shown in FIG. 4C. Thus, consideration of the energy of colored bonds, and even the placement of different colored bonds on distinct voxels, are important for promoting the assembly pathway, lattice growth, and minimizing defects.

Notably, however, the S(q) provides confirmation of the designed lattice of chiral NP motifs, as shown in FIG. 6B. Further, the embodiment corroborated the correspondence between the assembled and designed lattice through a real-space 3D imaging. As shown in FIG. 6C, serial sectioning SEM of a silicated lattice in conjunction with image reconstruction allows to decipher the hierarchical organization of spiraling NP motifs within the 3D lattice. Several views of the 3D reconstruction have been compared to ideal projections of the target structure, and the comparison reveals real-space images of the assembled structures that are strikingly similar to the target designed organization of stacked, spiral NP structures.

The presented systems and methods of the disclosed matter provide techniques for the assembly of arbitrarily designed and hierarchically organized 3D nanoscale architectures that can incorporate multiple types of nanocomponents. By utilizing encoded building blocks, voxels, for placement of nanocomponents, and for creating large scale 3D motifs, mesovoxels, for their assembly into 3D lattices utilizing intrinsic symmetries within the target structures to minimize the amount of information required for encoding the desired structures, it enables the fully prescribed nanofabrication of diverse 3D nanomaterials using bottom-up methods for a myriad of potential applications in optics, energy materials, and information processing.

What we claim is:

1. A system for encoding an assembly of three-dimensional (3D) hierarchically ordered nanoparticle architectures, comprising:
a non-transitory storage medium having instructions of an encoding program stored thereon,
one or more data processing apparatus configured to run the instructions of the encoding program to perform operations comprising:
a. identifying a target structure for the nanoparticle architecture;
b. determining chromatic bonds for each voxel
c. encoding the target structure by coordinating chromatic bonds within sets of voxels to create at least a mesovoxel; and
d. generating the assembly by using a symmetry of the mesovoxels.

2. The system of claim 1, further comprising an additive experimental device, wherein the one or more data processing apparatus is configured to run the instructions of the encoding program to output a structural indicator to the experimental device for verifying the assembly of 3D nanoparticle architectures.

3. The system of claim 2, wherein the experimental device includes an apparatus of small-angle X-ray scattering.

4. The system of claim 1, wherein the assembly comprises at least a lattice, which is characterized by lattice topology and lattice parameters.

5. The system of claim 1, wherein the symmetry includes a rotation symmetry and/or mirror symmetry.

6. The system of claim 1, wherein creating a mesovoxel includes determining number of voxels, internal colors, and external colors.

7. A method for encoding an assembly of three-dimensional (3D) hierarchically ordered nanoparticle architectures, comprising:
a. identifying a target structure for the nanoparticle architecture;
b. determining chromatic bonds for each voxel;
c. encoding the target structure by coordinating chromatic bonds within sets of voxels to create at least a mesovoxel; and
d. generating the assembly by using a symmetry of the mesovoxels.

8. The method of claim 7, wherein the chromatic bonds include the internal and external bonds of each voxel.

9. The method of claim 7, wherein the voxel is determined by using addressable interactions, where each bond has a specific encoding associated with both type and energy of interaction, while interactions of different colors are non-interacting.

10. The method of claim 7, wherein the voxel includes a DNA frame, for coordinating the placement of voxels within the mesovoxel.

11. The method of claim 7, wherein the voxel is configured as a DNA origami octahedron.

12. The method of claim 7, wherein the target structure includes 1D strings, 2D planes, face-perovskite lattices, and cubic lattices.

13. The method of claim 7, further comprising:
implementing an experimental verification of nanoparticle architectures using x-ray scattering and electron microscopy.

14. A method for designing a three-dimensional (3D) lattice of nanoparticles with nanoscale and photonic length scales, comprising:
a. identifying photonic regimes including periodicity, spacing, and separation of nanoparticle in the 3D lattice;
b. determining chromatic bonds for each voxel based on the photonic regimes;
c. encoding the nanoparticle architectures by coordinating chromatic bonds within sets of voxels to create at least a mesovoxel; and
d. assembling the 3D lattice with coupled nanoscale and photonic lengthscales by using a symmetry of the mesovoxels.

15. The method of claim 14, wherein the symmetry includes a mirror plane symmetry, where the mirror plane bisects the voxels at the midpoint between the two planes of nanoparticles-filled planes, enabling the use of two colors for coordinating voxels in the Z-direction.

16. The method of claim 14, wherein the mesovoxel consists of 6 unique voxels, 1 internal colored bond, and 5 external-colored bonds.

17. A method for designing a 3D lattice of ordered spiral nanoparticles motifs, comprising:
a. identifying a screw axis symmetry along Z-direction;
b. designing a mesovoxel including N₁ voxels displaying N₂ external colors, wherein N₁, N₂ is a positive integer;
c. rotating a N₃×N₃ voxel arrangement in the XY plane around the screw-axis along the Z-direction to generate a N₃×N₃ spiral, wherein N₃ is a positive integer;
d. incorporating nanoparticles into the voxels in the XY plane with specific particle placement and internal mesovoxel coloring;
e. executing a rotation through external mesovoxel binding with coloring of vertices in the Z-direction possessing N₄ colors with respective complements thereof on neighboring voxels, wherein N₄ is a positive integer;
f. translating the N₃×N₃ spiral in the XY plane using further external mesovoxel coloring to enable encoding a 3D lattice of ordered spiral nanoparticles motifs.

18. The method of claim 17, wherein N₁<4, and N₂<12.

19. The method of claim 17, wherein the ordered 3D lattice is arranged to generate plasmonic and light-emitting chiral clusters for light manipulation.

20. The method of claim 17, wherein the assembled 3D lattice is arranged to generate a crystal of periodic, spiraling assemblies with a screw axis along the Z-axis.
